(19) United States
(12) Patent Application Publication
(10) Pub. No.: US 2013/0065930 A1
(43) Pub. Date: Mar. 14, 2013

(54) TOPICAL COMPOSITION

(71) Applicants: Geena MALHOTRA, Mumbai (IN); Amar Lulla, Mumbai (IN); Martin Benedict George Donnelly, Nottingham (GB)

(72) Inventors: Geena MALHOTRA, Mumbai (IN); Amar Lulla, Mumbai (IN); Martin Benedict George Donnelly, Nottingham (GB)

(73) Assignee: Omnipharm Limited, Nottingham (GB)

(21) Appl. No.: 13/437,851

(22) Filed: Apr. 2, 2012

Related U.S. Application Data
(63) Continuation of application No. 13/201,845, now abandoned.
(60) Provisional application No. 61/161,361, filed on Mar. 18, 2009.

(30) Foreign Application Priority Data
Feb. 16, 2009 (IN) 333/MUM/2009

(51) Int. Cl. A61K 31/415; A61P 33/00

(52) U.S. Cl. 514/407

(57) ABSTRACT

A topical composition comprising: fipronil or a pharmaceutically acceptable salt thereof; at least one surfactant and at least one crystallization inhibitor for the treatment and protection of animals which are infested with parasites.

### CROSS REFERENCE TO RELATED APPLICATIONS

This application is a Continuation of Ser. No. 13/201,845, filed Aug. 16, 2011, which is a U.S. National Phase patent application of PCT/GB2010/000263, filed Feb. 15, 2010, which claims priority to Indian patent application Serial No. 333/MUM/2009, filed Feb. 16, 2009, and U.S. Provisional patent application Ser. No. 61/161,361, filed Mar. 18, 2009, all of which are hereby incorporated by reference in the present disclosure in their entirety.

### FIELD OF INVENTION

The present invention relates to a topical composition for the treatment and protection of animals which are infested with parasites or likely to be infested with them. In particularly, the aim of the invention is to provide an antiparasitic composition for treatment and protection of animals.

### BACKGROUND AND PRIOR ART

Pets are often infested with one or more of the parasites such as cat and dog fleas (Ctenocephalides felis, Ctenocephalides sp. and the like), ticks (Rhipicephalus sp., Ixodes sp., Dermacentor sp., Amblyoma sp. and the like), galls (Demodex sp., Sarcoptes sp., Otodectes sp. and the like).

Fleas cause an animal a great deal of stress and are harmful to its health. Moreover, fleas are also vectors of pathogenic agents, such as dog tapeworm (Dipylidium caninum), and can also attack man. Similarly, ticks can also cause an animal stress and be harmful to its health. They can also be harmful to humans. However, the most serious problem of ticks is that they are the vector of pathogenic agents which may affect the animal as much as humans.

Among the major diseases which need to be avoided, mention may be made of borrelioses (Lyme disease caused by Borrelia burgdorferi), babesioses (or piroplasmoses caused by Babesia sp.) and rickettsioses (also known as Rocky Mountain spotted fever). Ticks can also release toxins with paralysing and inflammatory properties, these toxins occasionally being fatal. Lastly, galls are particularly difficult to combat since there are very few active substances which act on these parasites, and they require frequent treatment.

At present, there are number of more or less active insecticides are available. However, drug resistance is often associated with their use, as is the case, for example, with carbamates, organophosphorus compounds and pyrethroids.

A new family of insecticides based on 1-N-phenylpyrazoles has been described in WO87/03781, EP295117 and EP352944. The compounds of the families defined in these patents are extremely active and one of these compounds, 1-[2,6-CI₂-4-CF₃-phenyl]-3-CN-4-[SO—CF₃]-5-NH₂ pyrazole, the common name of which is Fipronil, has proved to have a very broad spectrum activity including parasitic activity and is particularly effective, not only against crop parasites but also against ectoparasites of mammals and in particular, but not exclusively, fleas and ticks.

Commercially available formulations of Fipronil are FRONTLINE® from Merial, Inc, TREMIDOR®, TOP SPOT® and ADONIS® from Aventis CropScience S.A., Lyon, France.

U.S. Pat. No. 6,395,765 (to Merial Lyons, filed on Sep. 25, 1996) discloses a topical composition in the form of a ready-to-use solution comprising Fipronil.

U.S. Pat. No. 6,482,425 (to Merial Lyons, filed on Mar. 17, 1999) discloses a spot-on composition containing Fipronil and an endectocidal parasiticide of the macrocyclic class of compounds selected from the group consisting of avermectin, abamectin and doramectin;

U.S. Pat. No. 6,797,724 (to Merial Lyons, filed on Apr. 11, 2002) discloses a direct pour-on skin solution, comprising compounds that degrade; e.g. biodegrade, photodegrade or chemically degrade, to phenylpyrazoles and other excipients.

EP0296381 (to Bayer AG, filed on Dec. 28, 1988) describes pyrazole compounds having insecticidal activity in the field of agriculture, public health and veterinary medicine. Boophilus microplus is one of the many targets mentioned.

Other methods for administering pyrazole compounds include placing the therapeutic agent in a solid or liquid matrix for oral delivery. These methods include chewable drug-delivery formulations (WO2004016252 to Merial Limited, filed on Aug. 14, 2003). The problem associated with oral formulations is that the therapeutic agent often provides an unpleasant taste, aroma, or mouth-feel to the formulation, which cause, especially with animals, the oral formulation to be rejected.

Patent Application AU16427/95 mentions the combination of a substituted 1-N-pyrazole derivative of this type with avermectins, ivermectin or moxidectin, among a very large number of insecticides or parasiticides of various types, including Fipronil, however, without giving information on a composition comprising such a combination and without establishing a distinction regarding the susceptible targets by specific combinations, among the innumerable parasites which can potentially be attacked.

Most of the prior art anti-parasitic formulations employs crystallization inhibitors (such as low molecular weight polyvinylpyrrolidone, copolymers of vinyl acetate and vinyl pyrrolidone and polyoxyethylenated sorbitan esters).

Without being bound by any theory, it is known that surfactants may also be used in combination with crystallization inhibitors, selection of a specific surfactant in combination with a specific crystallization inhibitor is challenging because of incongruity between various surfactants and crystallization inhibitors in serving its intended purpose.

Improper selection of the surfactant may reduce the inhibiting effect of crystallization inhibitor and hamper its intended purpose in the product. For example, both non-ionic PEG-10-olylether and hexadecylpyridinium cations reduce the inhibiting effect of PVP on crystallization. (K. H. Ziller et al., Control of crystal growth in drug suspensions, 1988, Drug Development and Industrial Pharmacy, 14 (15-17), 2341-2370).

Also, in the presence of some polymers, surfactant molecules may associate with polymers to form surfactant-polymer aggregates (complexes). (Fang Li. Et. al., 1998, Colloid Polym Sci 276:1-10).

Some surfactants however, may hinder protective action of crystallization inhibitors on drug almost completely. For example, the surfactant ‘hexadecyl sulphate’ aggregates with PVP in aqueous phase and thus prevents PVP from establishing protective layers on the drug particles.

In one particular example using acetaminophen (Saito, S., T. Taniguchi and K. Kitamura. J. Coll. Int. Sci. 37 (1971), 154-164), surfactants may disturb the structure of protective layer of crystallization inhibitors at the drug surfaces leading to further crystallization of acetaminophen.

The said combination should also be miscible and suitable with the solvent system of the anti-parasitic composition.

Hence, there still exists a continuing need to select a proper combination of crystallization inhibitor and surfactant in the antiparasitic formulations so as to overcome the shortcomings of prior art.

Furthermore, fipronil formulations are disclosed in GB 2 331 242 A, which also discloses the combination of Fipronil with other parasiticides. Furthermore, GB 2 317 264 A discloses Fipronil formulations additionally comprising an IGR (insect growth regulator) compound, e.g. Methoprene.

However, a problem with these known formulations is their relatively low flashpoints, i.e. the lowest temperatures at which they can form an ignitable mixture in air. The Material Safety Data Sheet for the FRONTLINE® PLUS FOR DOGS formulation indicates the flashpoint for this formulation to be 36° C. (97° F.). A liquid which forms an ignitable mixture at 36° C. presents a safety risk during use in the home, and during manufacture, distribution and storage, because temperatures in many countries exceed this level during summer. There is therefore a need for effective parasiticidal formulations comprising Fipronil which have higher flashpoints, and therefore improved safety profiles, but which still retain parasiticidal efficacy.

### OBJECTS OF THE INVENTION

The object of the invention is to provide a topical composition which, when applied locally, will subsequently spread over the animal's entire body and then dry, while at the same time avoiding any phenomenon of crystallization over a significant time period.

Another object of the invention is to provide topical antiparasitic compositions for the treatment and protection of animals, these compositions being of great efficacy while at the same time being easy to use.

Yet another object of the invention is to provide a topical composition which is easy to use on any type of domestic animal, irrespective of its size and the nature of its coat.

Yet another object of the invention is to provide a topical composition which is effective and which is not required to be sprinkled over the animal's entire body.

Yet another object of the invention is to provide a topical composition which, after drying, gives good appearance and feel of non-sticky coat after application.

Another object is to provide a composition comprising fipronil having improved safety while maintaining parasiticidal efficacy.

Still another object of the present invention is to provide a topical composition with ease of manufacture.

### SUMMARY OF THE INVENTION

According to the present invention, there is provided a topical composition comprising Fipronil or a pharmaceutically acceptable salt thereof; at least one crystallization inhibitor and at least one surfactant and at least one pharmaceutically acceptable excipient.

In one embodiment, the topical composition further includes at least one other anti-parasitic agent other than fipronil.

In an embodiment, the topical composition of the present invention includes fipronil and S-methoprene or its salt.

In another embodiment, the present invention provides method of treating a parasitic infestation in an animal.

In a further embodiment, the present invention provides a method of improving the stability of a topical composition comprising fipronil and optionally with at least one antiparasitic agent.

In a further specific embodiment, the present invention provides a method of improving the stability of a topical composition comprising fipronil and S-methoprene.

The invention further provides a process for making the topical composition.

### DETAILED DESCRIPTION

As described hereinbefore, there is a continuing need of an antiparasitic topical composition which is efficacious, easy to use, having improved safety profile and avoids crystallization of active over a significant time period.

It has been surprisingly found that a suitable combination of surfactant such as polyoxyethylenated castor oil derivatives and crystallization inhibitors resulted in inhibition of crystallization of active over a significant time period.

The term “topical composition” (or synonymously “topical parasiticidal composition” or formulation) as used herein refers to a composition that can be topically applied to mammalian keratinous tissue. Examples of such compositions include, but not limited to dispersion, solution, emulsion, suspension, ointment, cream, paste, gel, lotion.

The amount of fipronil or derivative thereof, in the composition is preferably from 5% to 20% by weight, more preferably from 5% to 15% by weight and most preferably from 8% to 12% by weight of the topical composition.

The term “pharmaceutically acceptable salt” (or synonymously “salt”) as used herein refers to a solvates, hydrates, enantiomers, derivatives, polymorphs, prodrugs. In particular, any veterinarily acceptable derivative of fipronil can be used.

Polyoxyethylene castor oil derivatives are complex mixtures of various hydrophobic and hydrophilic components. These compounds are non-ionic surfactants which are approved for use in oral, topical, and parenteral pharmaceutical formulations. The polyoxyethylene castor oil derivatives are mainly used as emulsifying and solubilizing agents for the production of aqueous liquid preparations containing oils or hydrophobic drugs. Examples of these compounds which are suitable for use in the present invention may be selected from, but not limited to polyoxyethylene 5 castor oil (Acconon CA-5), polyoxyethylene 9 castor oil (Acconon CA-9), polyoxyethylene 15 castor oil (Acconon CA-15), polyoxyethylene 35 castor oil (Cremophor EL, Cremophor ELP, Etocas 35), polyoxyethylene 40 castor oil, polyoxyl 40 hydrogenated castor oil (Cremophor RH 40, Emulgin HRE 40), polyoxyl 40 hydrogenated castor oil (Emulgin HRE 60). Preferably, the surfactant is a polyoxyethylenated castor oil derivative.

The amount of the surfactant, especially the polyoxyethylene castor oil derivative, in the topical composition preferably ranges from 1 to 20% by weight, more preferably from 2% to 15% by weight, most preferably from 2% to 10% by weight, of the topical composition

It will be well appreciated to the person skilled in the art that the anti-parasitic topical composition according to the present invention may be achieved by using a surfactant selected from, but not limited to, non-ionic surfactants such as polyoxyethylenated esters of sorbitan (e.g. polysorbate 20, polysorbate 60 & polysorbate 80); propylene glycols and fatty acid esters of propylene glycol (e.g. propylene glycol monocaprylate, propylene glycol monolaurate); oleoyl macrogol glycerides (e.g. Labrafil); Caprylocaproyl macrogol glycerides (e.g. Labrasol); polyethylene glycols (e.g. PEG 600, PEG 6000); copolymers of ethylene oxide & propylene oxide (e.g. Poloxamers) or combinations thereof.

A crystallization inhibitor is an agent which prevents crystallization of the drug from the topical composition in the container or the hair or skin of the animal.

The topical composition according to the present invention may comprise more than one crystallization inhibitor. The or each crystallization inhibitor preferably satisfies the following test having steps (i)-(iii): (i) 0.5 ml of a topical composition of the invention comprising the at least one crystallisation inhibitor in an amount of 10% by weight is deposited in an open Petri dish at 20° C.; (ii) the deposited composition is observed at 20 minute intervals; and (iii) no crystals are observed with the naked eye within 3 hours of depositing the composition.

More preferably the amount of crystallization inhibitor in the topical composition is from 1 to 20% by weight, more preferably 2% to 15% by weight, most preferably 2% to 10% by weight of the topical composition.

The crystallization inhibitor used in the present invention, may be selected from, but not limited to polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose; acrylic derivatives such as methacrylates, lauryl-substituted betaine compounds. Polyethylene glycols are preferred.

Alternatively, or in addition to the above, the crystallization inhibitor may be a nonionic, cationic and/or anionic surfactant.

According to another preferred embodiment, the topical composition further comprises one or more additional anti-parasitic agents selected from, but not limited to insect growth regulators such as S-methoprene, pyriproxyfens, hydroprene, cyromazine, lufenuron and 1-(2,6-difluorobenzoyl)-3-(2-fluoro-4-(trifluoromethyl)phenylurea; compounds which mimics juvenile hormones such as azadirachtin, diofenolan, fenoxycarb, hydroprene, kinoprene, methoprene, pyriproxyfen, tetrahydroazadirachtin, and 4-chloro-2-(2-chloro-2-methyl-propyl)-5-(6-iodo-3-pyridylmethoxy)-pyridizine-3(2H)-one; chitin-synthesis inhibitors such as chlorfluazuron, cyromazine, diflubenzuron, fluazuron, flucycloxuron, flufenoxuron, hexaflumuron, lufenuron, tebufenozide, teflubenzuron, triflumuron, 1-(2,6-difluorobenzoyl)-3-(2-fluoro-4-(trifluoromethyl)phenylurea, 1-(2,6-difluoro-benzoyl)-3-(2-fluoro-4-(1,1,2,2-tetrafluoroethoxy)-phenylurea and 1-(2,6-difluoro-benzoyl)-3-(2-fluoro-4-trifluoro-methyl)phenylurea or their pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof. An exemplary combination is fipronil and S-methoprene.

In one embodiment, the amount additional anti-parasitic agent in the composition is from 1% to 25% by weight, preferably from 2% to 20% by weight, more preferably from 5% to 15% by weight, and most preferably from 8% to 12% by weight of the composition.

The topical composition preferably further includes an organic solvent. The organic solvent preferably has a dielectric constant of from 10 to 40, more preferably from 10 to 35, and most preferably from 15 and 30. The content of the organic solvent (and any cosolvent, as mentioned below) in the total composition preferably represents the remainder to about 100% by weight of the composition.

The topical composition preferably further includes an organic cosolvent, which preferably has a boiling point lower than 100° C.; preferably lower than 80° C.; and preferably has dielectric constant of from 10 to 40, most preferably of from 15 to 30. The w/w ratio of organic cosolvent to organic solvent (when present) is preferably present in the composition is about 1/15 to about 1/2. The cosolvent is preferably volatile in order to promote drying and is miscible with water and/or with the solvent.

The organic solvent used in the topical composition according to the present invention, may be selected from, but not limited to acetone, acetonitrile, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, benzyl alcohol, liquid polyoxyethylene glycols, propylene glycol, 2-pyrrolidone, in particular N-methylpyrrolidone, diethylene glycol monoethyl ether, dipropylene glycol n-butyl ether, dipropylene glycol monomethyl ether, ethylene glycol, diethyl phthalate, or a mixture of at least two of these solvents. The preferred solvent is selected from glycol ethers, in particular diethylene glycol monoethyl ether, dipropylene glycol n-butyl ether and dipropylene glycol monomethyl ether. Diethylene glycol monoethyl ether (eg Transcutol P) is most preferred.

However, in a preferred embodiment, the organic solvent is not a C₁ to C₆ alcohol cosolvent.

In a particularly preferred embodiment, the organic cosolvent is a C₁ to C₆ alcohol. Preferred examples of the organic cosolvent include methanol, ethanol, propanol, isopropanol, butanol and combinations thereof. Ethanol is the most preferred cosolvent.

In the context of present invention, the cosolvent preferably comprises up to 20% by weight, more preferably up to 15% by weight, and most preferably up to 10% by weight of the composition. Preferably there is at least 1% by weight of the cosolvent in the composition. More preferably there is at least 2% by weight of the cosolvent in the composition.

As antioxidant, standard agents may be used in particular, such as butylated hydroxyanisole (BHA), butylated hydroxytoluene (BHT), ascorbic acid, sodium metabisulphite, propyl gallate, sodium thiosulphate, a mixture of these antioxidants.

The composition preferably includes an antioxidant. The amount of antioxidant in the topical composition according to the present invention preferably ranges from 0.005 to 1% by weight of the composition, more preferably 0.005 to 0.05% by weight of the composition. In a particularly preferred embodiment, the composition comprises about 0.03% by weight of the antioxidant.

Examples suitable antioxidants include butylated hydroxylanisole, butylated hydroxyltoluene, alpha tocopheral, ascorbic acid, ascorbyl palmitate, fumeric acid, malic acid, citric acid, sodium ascorbate, sodium metabisulfate, n-propyl gallate, monothioglycerol and combinations thereof. In one embodiment, the antioxidant is butylated hydroxylanisole, butylated hydroxyltoluene or combinations thereof. In one embodiment, the at least one antioxidant comprises or consists of butylated hydroxylanisole (preferably in amount of about 0.02% by weight of the composition) and butylated hydroxyltoluene (preferably in amount of 0.01% by weight of the composition).

Although water is not preferred, the topical composition according to the present invention may optionally contain water in a proportion of from 0 to 30% by weight of the composition, preferably, from 0 to 5% by weight of the composition.

Oils may advantageously be utilized in the topical compositions of the invention. For example, heavy oils such as mineral or vegetable including corn, soybean and peanut oil, and petroleum fractions such as paraffinic or aromatic hydrocarbons may be used.

The composition formulated according to the invention up achieves, the absence of crystallization on the hair or skin and of maintenance of the cosmetic appearance of the coat, that is to say a tendency not to stick together or to have a sticky appearance, despite high concentration of active substance.

The parasiticidal compositions of the present invention surprisingly have a flashpoint of greater than 36° C. (97° F.). Particularly, compositions of the present invention have been shown to have flashpoints from about 45° C. to about 55° C. and are therefore safer than the known compositions of the prior art. The compositions of the present invention also retain parasiticidal efficacy.

The term “flash point” as used herein according to the present invention means the minimum temperature (at least 40° C.). at which a topical composition can form an ignitable mixture. The flash point can be determined by various methods known in the art. The flash point of the topical compositions according to the present invention was determined by well-known Abel Cup method.

The topical composition according to the invention intended for animals may be applied by deposition on the skin (“spot on” or “pour on” application); this may be a localized application in particular at one or two points and preferably localized between the animal's shoulders. After deposition, the composition diffuses, in particular over the animal's entire body, and then dries, without crystallizing or changing the appearance (in particular absence of any whitish deposit or of any dusty appearance) or the feel of the coat. The composition is typically applied over a surface area of up to 10 cm², normally from 5 and 10 cm².

The topical composition according to the invention is particularly advantageous on the grounds of its efficacy, its speed of action and the pleasant appearance of the animal's hair after application and drying. Once deposited, the composition diffuses over the mammal's body and dries without crystallizing or modifying the appearance or feel of the fur.

The invention also provides the use of a surfactant and a crystallization inhibitor to improve the stability of a composition comprising fipronil, or a pharmaceutically acceptable salt thereof.

Alternatively, the invention provides a method of improving the stability of a composition comprising fipronil, or a pharmaceutically acceptable salt thereof, comprising using an effective amount of a surfactant and a crystallization inhibitor.

The invention further provides the use of a crystallization inhibitor, an organic solvent and an organic cosolvent to raise the flashpoint of a composition comprising fipronil, or a pharmaceutically acceptable salt thereof.

Alternatively, the present invention provides a method of raising the flashpoint of a topical composition comprising fipronil, or a pharmaceutically acceptable salt thereof, comprising using an effective amount of a surfactant, a crystallization inhibitor, an organic solvent and an organic cosolvent.

The term “effective amount” as used herein refers to the amount necessary to bring about the desired results according to the present invention.

The present invention also provides a process to manufacture the antiparasitic topical composition, which process comprises—

(1) dissolving a surfactant and/or a crystallization inhibitor to an organic solvent;
(2) adding the or each active ingredient to the above mixture under stirring, followed by addition of cosolvent and finally making up the weight with remainder of organic solvent and mixing.

After step (1) and before step (2), other excipients may be added, in particular the antioxidant.

In a preferred embodiment according to the present invention, there is also provided use of the topical composition for treating and/or protecting (preventive care) of animals against parasites (especially ectoparasites, such as ticks or fleas), according to which an anti-parasitically effective volume of a composition according to the invention is applied to a limited area of the animal, as is described above. The application is advantageously made at two points and/or on the animal's back between the shoulders.

The purpose of application of the topical composition of the present invention may be non-therapeutic, when it concerns cleaning the animal's hair and skin by eliminating the parasites present as well as their residues and excreta. The animal thus has a coat which is pleasant to look at and to feel. This also makes it possible to prevent the establishment of fleas in the house.

The purpose of application of the topical composition of the present invention may also be therapeutic when it concerns treating a parasitosis which has pathogenic consequences.

The volume applied may be about 0.3 to 1 ml, preferably about 0.5 ml for cats; and about 0.3 to 3 ml for dogs, or to any animal depending on its weight. It is to be understood that these dosage values are average values which may vary because the composition will be administered to mammals having relatively different body weights. Consequently, the doses applied may be smaller or larger than the doses provided above.

The volume of composition applied preferably corresponds to a dose of the antiparasitic agent from 0.1 to 80 mg, preferably from 0.3 and 60 mg, more preferably 1 to 40 mg, still more preferably 1 to 30 mg, and most preferably 5 to 15 mg per kg of body weight of the animal. As described above, the anti-parasitic agent may consist of fipronil, or it may be fipronil in combination with one or more other anti-parasitic agents, such as S-methoprene.

Treatment of mammals, in particular cats and dogs, with the composition of the present invention may be carried out, for example, every one, two or three months.

The following examples are for the purpose of illustration of the invention only and are not intended in any way to limit the scope of the present invention.

### Example 1

### Topical Composition of Fipronil

| Sr. | | Qty in % w/w | |
|---|---|---|---|
| No. | Ingredients | Formula 1 | Formula 2 |
| 1 | Fipronil | 9.7 | 9.7 |
| 2 | Ethanol | 5 | 7.5 |
| 3 | Butylated Hydroxy Anisole | 0.02 | 0.02 |
| 4 | Butylated Hydroxy Toluene | 0.01 | 0.01 |
| 5 | Polyethylene glycol 60 | 5 | 5 |
| | Hydrogenated castor oil | | |
| | (Nikkol HCO 60) | | |
| 6 | Polyethylene Glycol 1000 | 5 | 7.5 |
| | (Transcutol P) | | |
| 7 | Diethyl glycol monoethyl ether | q. s. to 100 gm | q. s. to 100 gm |

### Process:

(1) Polyethylene glycol 60 hydrogenated castor oil & polyethylene glycol 1000 were added in 50% w/w batch quantity of diethyl glycol monoethyl ether.
(2) The bulk of step (1) was warmed to dissolve both polyethylene glycol 60 hydrogenated castor oil & polyethylene glycol 1000 followed by addition of butylated hydroxy toluene and butylated hydroxy anisole and the mixture was allowed to cool.
(3) Fipronil was added to the above mixture under stirring followed by addition of ethanol and finally the weight of the composition was made with diethyl glycol monoethyl ether and mixed.

The flashpoints of the above composition was measured and found to be 46° C.

### Example 2

### Topical Composition of Fipronil and S-Methoprene

| | | Formulation for CAT | | Formulation for DOG | |
|---|---|---|---|---|---|
| Sr. | | Formula 1 | Formula 2 | Formula 1 | Formula 2 |
| No. | Ingredients | Qty in % w/w | Qty in % w/w | Qty in % w/w | Qty in % w/w |
| 1 | Fipronil | 9.80 | 9.80 | 9.80 | 9.80 |
| 2 | S-Methoprene | 11.80 | 11.80 | 8.80 | 8.80 |
| 3 | Ethanol | 5.00 | 7.50 | 5.00 | 7.50 |
| 4 | Butylated Hydroxy Anisole | 0.02 | 0.02 | 0.02 | 0.02 |
| 5 | Butylated Hydroxy Toluene | 0.01 | 0.01 | 0.01 | 0.01 |
| 6 | Polyethylene glycol 60 | 5.00 | 5.00 | 5.00 | 5.00 |
| | Hydrogenated castor oil | | | | |
| | (Nikkol HCO 60) | | | | |
| 7 | Polyethylene Glycol 1000 | 5.00 | 7.50 | 5.00 | 7.50 |
| 8 | Diethylene glycol monoethyl | q. s. to 100 gm | q. s. to 100 gm | q. s. to 100 gm | q. s. to 100 gm |
| | ether (Transcutol P) | | | | |

### Process:

(1) Polyethylene glycol 60 hydrogenated castor oil & polyethylene glycol 1000 was added in 50% w/w batch quantity of diethyl glycol monoethyl ether.
(2) The bulk of step (1) was warmed to dissolve both the polyethylene glycol 60 hydrogenated castor oil & polyethylene glycol 1000 followed by addition of butylated hydroxy toluene and butylated hydroxy anisole and the mixture was allowed to cool.
(3) Fipronil was added to the above mixture under stirring followed by addition of s-methoprene & ethanol and finally the weight of the composition was made with diethyl glycol monoethyl ether and mixed.

The flashpoints of the above composition of formula 1 for cat and formula 1 for dog were measured and found to be 52° C. and 50° C. respectively

The compositions of the present invention therefore have a reduced propensity to form ignitable mixtures with air. They therefore provide a safer composition for use, storage, distribution and manufacture.

### Example 3

Stability studies were conducted on the topical antiparasitic compositions of the present invention, the results of which are tabulated below.

Stability study data for Fipronil Spot-on composition (9.7% w/w of Fipronil, 5% w/w of ethanol, 5% w/w of polyoxyethylene castor oil & 5% w/w PEG 1000)

| | | Impurity | |
|---|---|---|---|
| | | % Single | |
| Condition | Assay | max | % Total |
| Initial | 102.9 | 1.037 | 1.3 |
| 3M 25° C./60% Rh | 102.59 | 1.042 | 1.405 |
| 3M 30° C./70% Rh | 103.78 | 1.044 | 1.416 |
| 3M 40° C./75% Rh | 102.04 | 1.044 | 1.452 |

The stability study results show that the anti-parasitic composition of the present invention is stable over a three month storage period.

### Example 5

### Parasiticidal Activity

The compositions of Examples 1-3 have been shown in trials to have parasiticidal activity.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a preservative” includes a single preservative as well as two or more different preservatives; reference to a “surfactant” refers to a single surfactant or combination of two or more surfactants, and the like.

What is claimed is:

1. A topical composition comprising: fipronil or a pharmaceutically acceptable salt thereof; at least one surfactant and at least one crystallization inhibitor, wherein the flashpoint of the composition is at least 40° C.

2. A topical composition according to claim 1, wherein the flash point of the composition ranges from about 45° C. to about 55° C.

3. A topical composition according to claim 1, wherein surfactant is present in an amount ranging from 1 to 20% by weight of the composition.

4. A composition according to claim 1, wherein crystallization inhibitor is present in an amount ranging from 1 to 20% by weight of the composition.

5. A topical composition according to claim 1, wherein the surfactant comprises a polyoxyethylenated ester of sorbitan, such as polysorbate 20, polysorbate 60 and/or polysorbate 80; a polyoxyethylene castor oil derivative a propylene glycol; a fatty acid ester of propylene glycol such as propylene glycol monocaprylate, propylene glycol monolaurate; an oleoyl macrogol glyceride; a caprylocaproyl macrogol glyceride; a polyethylene glycol; a copolymers of ethylene oxide & propylene oxide; or a combination thereof.

6. A topical composition according to claim 1, wherein the surfactant is a polyoxyethylene castor oil derivative.

7. A topical composition according to claim 1, wherein the surfactant is a polyethylene glycol hydrogenated castor oil.

8. A topical composition according to claim 1, wherein the crystallization inhibitor comprises polyvinylpyrrolidone, a polyvinyl alcohol, a copolymer of vinyl acetate and vinylpyrrolidone, a polyethylene glycol, benzyl alcohol, mannitol, glycerol, sorbitol, a polyoxyethylenated sorbitan ester; lecithin, sodium carboxymethylcellulose; an acrylic derivatives such as a methacrylate; an anionic, cationic or anionic surfactant; or a combination thereof.

9. A topical composition according to claim 1, wherein the crystallization inhibitor is polyethylene glycol.

10. A topical composition according to claim 1 further comprises at least one organic solvent and at least one organic cosolvent.

11. A topical composition according to claim 1, wherein the organic cosolvent is a C₁ to C₆ linear or branched alcohol.

12. A topical composition according to claim 1, wherein the organic cosolvent is methanol, ethanol or isopropanol.

13. A topical composition according to claim 1, wherein the organic cosolvent is present in an amount ranging from 2% to 15% by weight of the composition.

14. A topical composition according to claim 1, wherein the organic solvent is not a C₁ to C₆ alcohol cosolvent.

15. A topical composition according to claim 1 wherein the organic solvent is diethylene glycol monoethyl ether.

16. A topical composition comprising fipronil, at least one antiparasitic agent other than fipronil, or pharmaceutically acceptable salt thereof; at least one surfactant and at least one crystallization inhibitor, wherein the flashpoint of the composition is at least 40° C.

17. A topical composition according to claim 16, wherein the additional anti-parasitic agent comprises pyriproxyfens, hydroprene, cyromazine, lufenuron and 1-(2,6-difluorobenzoyl)-3-(2-fluoro-4-(trifluoromethyl)phenylurea, azadirachtin, diofenolan, fenoxycarb, hydroprene, kinoprene, S-methoprene, pyriproxyfen, tetrahydroazadirachtin, and 4-chloro-2-(2-chloro-2-methyl-propyl)-5-(6-iodo-3-pyridylmethoxy)-pyridizine-3(2H)-one, chlorfluazuron, cyromazine, diflubenzuron, fluazuron, flucycloxuron, flufenoxuron, hexaflumuron, lufenuron, tebufenozide, teflubenzuron, triflumuron, 1-(2,6-difluorobenzoyl)-3-(2-fluoro-4-(trifluoromethyl)phenylurea, 1-(2,6-difluoro-benzoyl)-3-(2-fluoro-4-(1,1,2,2-tetrafluoroethoxy)-phenylurea and 1-(2,6-difluoro-benzoyl)-3-(2-fluoro-4-trifluoro-methyl)phenylurea.

18. A topical composition according to claim 16, wherein the antiparasitic agent is S-methoprene or pharmaceutically acceptable salt thereof.

19. A topical parasiticidal composition comprising: Fipronil in an amount of about 9.7%; ethanol in an amount of about 5.0% to about 7.5%; butylated hydroxylanisole in an amount of about 0.02%; butylated hydroxyltoluene in an amount of about 0.01%; polyethylene glycol 1000 in an amount of about 5.0% or about 7.5%; polyethylene glycol 60 hydrogenated castor oil in an amount of about 5.0% by weight of the composition; and diethylene glycol monoethyl ether in an amount to make up the final volume of the composition.

20. A topical parasiticidal composition comprising: Fipronil in an amount of about 9.8%; S-Methoprene in an amount of about 11.8%; ethanol in an amount of about 5.0% to about 7.5%; butylated hydroxylanisole in an amount of about 0.02%; butylated hydroxyltoluene in an amount of about 0.01%; polyethylene glycol 1000 in an amount of about 5.0% to about 7.5%; polyethylene glycol 60 hydrogenated castor oil in an amount of about 5.0% by weight of the composition; and diethylene glycol monoethyl ether in an amount to make up the final volume of the composition.

21. A topical parasiticidal composition comprising: Fipronil in an amount of about 9.8%; S-Methoprene in an amount of about 8.8%; ethanol in an amount of about 5.0% to about 7.5%; butylated hydroxylanisole in an amount of about 0.02%; butylated hydroxyltoluene in an amount of about 0.01%; polyethylene glycol 1000 in an amount of about 5.0% to about 7.5%; polyethylene glycol 60 hydrogenated castor oil in an amount of about 5.0% by weight of the composition; and diethylene glycol monoethyl ether in an amount to make up the final volume of the composition.

22. A topical composition according to claim 1 for use as a parasaticide.

23. A method of treating a parasitic infestation in an animal comprising administering an effect amount of a topical composition according to claim 1 to an animal in need thereof.

24. A method of improving the stability of a composition comprising fipronil, or a pharmaceutically acceptable salt thereof, comprising using an effective amount of a surfactant and a crystallization inhibitor.

25. A method of raising the flashpoint of a topical composition comprising fipronil, or a pharmaceutically acceptable salt thereof, comprising using an effective amount of a surfactant, a crystallization inhibitor, an organic solvent and an organic cosolvent.

26. A process of manufacturing the topical composition according to claim 1.

27. A topical composition substantially as herein described and illustrated with examples.
